(19) United States
(12) Patent Application Publication
(10) Pub. No.: US 2026/0034374 A1
(43) Pub. Date: Feb. 5, 2026

(54) Active Medical Device Having A Ceramic Housing Connected To A Feedthrough

(71) Applicant: Greatbatch Ltd., Clarence, NY (US)

(72) Inventors: Keith W. Seitz, Clarence Center, NY (US); Thomas Marzano, East Amherst, NY (US); Jonathan Calamel, Clarence, NY (US); Dallas J. Rensel, Sanborn, NY (US)

(21) Appl. No.: 18/817,598

(22) Filed: Aug. 28, 2024

Related U.S. Application Data
(60) Provisional application No. 63/536,158, filed on Sep. 1, 2023.

(51) Int. Cl. A61N 1/375; A61N 1/372; A61N 1/378

(52) U.S. Cl. CPC A61N 1/3758; A61N 1/37211; A61N 1/3754; A61N 1/3787

(57) ABSTRACT

An active medical device comprises a ceramic housing having an annular rim surrounding an opening. A housing ferrule sealed to the annular rim has a ferrule opening. A PCB assembly contained inside the housing comprises a printed circuit board supporting a plurality of electronic components that are connected to electrical terminations. An electrical power source inside the ceramic housing powers the electronic components. The insulator of a glass-to-metal seal (GTMS) supports a plurality of terminal pins, and the device side portion of a terminal pin is connected to a respective one of the electrical terminations of the PCB assembly. This connects a terminal pin to an electronic component of the PCB assembly. Then, the GTMS ferrule is sealed to the housing ferrule so that the GTMS closes the opening of the ceramic housing containing the PCB assembly.

### CROSS-REFERENCE TO RELATED APPLICATION

This application claims priority to U.S. Provisional Application Ser. No. 63/536, 158, filed on Sep. 1, 2023.

### BACKGROUND OF THE INVENTION

### 1. Field of the Invention

The present invention relates to the field of externally worn or implantable active medical devices. More particularly, the present invention relates to an active medical device (AMD) that is designed to deliver electrical stimulation to a patient or sense biological signals from body tissue.

### 2. Prior Art

A conventional active medical device has a metallic housing, typically made of titanium, that is connected to a molded header supporting a number of terminal blocks. The header terminal blocks are connected to terminal pins comprising a feedthrough. Proximal ends of the terminal pins are electrically connected to device electronics mounted on a printed circuit board (PCB) to form a PCB assembly contained inside the titanium housing. The housing also contains an electrochemical cell as an electrical power source for powering the electronics. Distal ends of the terminal pins are connected to the terminal blocks in the header. Then, the proximal electrical contacts of a lead are inserted into the header to detachably connect the electrical contacts to the terminal blocks. This connection establishes continuity from electrodes at the distal end of the lead to the device electronics of the PCB assembly contained inside the device housing. The lead electrodes are configured to send electrical pulses to body tissue in which they are implanted or to sense biological signals from the tissue.

For some medical applications, it is conventional to provide the electrical power source as an electrochemical cell having a primary chemistry. When the cell reaches end-of-life, the medical device is explanted and replaced with a new device. However, recent medical advancements have led to many medical devices being powered by secondary or rechargeable electrochemical cells. In a conventional titanium housing, this means that in addition to the terminal blocks being supported in the header, a charging coil is also housed in the device header. The charging coil and the terminal blocks residing in the header take up a substantial amount of space.

Therefore, there is an ongoing need for an AMD, whether implantable or intended to be worn externally, that is detachably connectable to a lead to provide both stimulation and sensing capability and that is powered by a rechargeable electrochemical cell that is connected to a charging circuit contained inside the device housing. Moving the charging coil out of the header and inside the device housing is expected to save a substantial amount of space in the molded header. A smaller medical device is easier to implant in a patient and would be expected to cause less trauma to the patient. A smaller medical device is also expected to be less bothersome to a patient.

In that respect, there is a desire to provide an active medical device powered by a rechargeable electrochemical cell with the charging coil connected to the charging circuit contained inside the device housing. This advancement is made possible by constructing the device housing from a ceramic material, for example, alumina.

### SUMMARY OF THE INVENTION

One of the attributes of the present invention is that the device housing is made from a ceramic material. This means that the charging coil can be moved out of the header and housed inside the ceramic housing. Unlike titanium and other metals that are used to make a device housing, a ceramic material permits RF or inductive energy to penetrate through the housing to reach the charging coil. The charging coil connected to a charging circuit are components of the PCB assembly contained inside the ceramic housing. The charging circuit is configured to convert RF or inductive energy received from the charging coil into a direct current voltage to charge the rechargeable electrical power source to power the electronic component of the PCB assembly.

Thus, the present invention relates to an active medical device that is configured to both provide electrical stimulation and sense biological signals to and from body tissue in which it is implanted or on which it is worn. The active medical device is selected from a cochlear implant, a piezoelectric sound bridge transducer, a neurostimulator, a brain stimulator, a brain sensor, a neurostimulator configured to stimulate the Vagus nerve, an optical sensor, a motion sensor, an acoustic sensor, a pressure sensor, an analyte sensor, an electromagnetic sensor, a cardiac pacemaker, a left ventricular assist device, an artificial heart device, an insulin drug pump, a chemotherapy drug pump, a pain medication pump, a bone growth stimulator, a urinary incontinence device, a spinal cord stimulator, an anti-tremor stimulator, an implantable cardioverter defibrillator, a congestive heart failure device, an external insulin pump, an external drug pump, and an external neurostimulator, an external ventricular assist device.

More particularly, the active medical device is comprised of a ceramic housing, preferably of alumina, having a housing sidewall extending to an annular rim surrounding a housing opening leading into the housing. A housing ferrule sealed to the annular rim of the ceramic housing has a ferrule opening. A printed circuit board (PCB) assembly is contained inside the ceramic housing. The PCB assembly comprises a printed circuit board supporting at least one electronic component that is configured to control functions of the active medical device, wherein the printed circuit board is provided with at least one electrical termination electrically connected to the at least one electronic component. Preferably, the printed circuit board has a plurality of electrical terminations and the insulator of the GTMS supports a plurality of terminal pins. One of the plurality of terminal pins is connected to one the plurality of electrical terminations of the PCB assembly.

Further, an electrical power source contained inside the ceramic housing is electrically connected to the at least electronic component of the PCB assembly. The electrical power source is selected from a capacitor, an alkaline cell, a primary lithium cell, a rechargeable lithium-ion cell, a Ni/cadmium cell, a Ni/metal hydride cell, a supercapacitor, and a thin film solid-state cell with a rechargeable chemistry being preferred.

The active medical device of the present invention also has a glass-to-metal seal (GTMS). The GTMS has a GTMS ferrule with a GTMS ferrule opening. An insulator comprising an insulator sidewall hermetically sealed to the GTMS ferrule in the ferrule opening extends to an insulator device side spaced from an insulator body fluid side. There is at least one via hole extending through the insulator to the device and body fluid sides, and at least one terminal pin is hermetically sealed to the insulator in the via hole. The terminal pin has a proximal or device side portion that extends outwardly beyond the insulator device side and a distal or body fluid side portion that extends outwardly beyond the insulator body fluid side. Further, with the terminal pin device side portion being electrically connected to the at least one electrical termination of the PCB assembly, the GTMS ferrule is sealed to the housing ferrule so that the GTMS closes the housing opening of the ceramic housing.

In the active medical device, the electrical power source is either supported on the printed circuit board or positioned adjacent to the PCB assembly to power the at least one electronic component. Further, a charging coil is connected to a charging circuit contained inside the ceramic housing, and the electrical power source is rechargeable. That way, the charging circuit is configured to convert RF or inductive energy received from the charging coil into a direct current voltage to charge the rechargeable electrical power source to power the at least one electronic component of the PCB assembly.

In another embodiment, the GTMS ferrule and the housing ferrule are made of titanium, and they are welded together.

In another embodiment, the printed circuit board is provided with a plurality of electrical terminations and the insulator of the GTMS supports a plurality of terminal pins. Then, one of the plurality of terminal pins is connected to one of the plurality of electrical terminations of the PCB.

Another embodiment of the active medical device that is configured to both provide electrical stimulation and sense biological signals to and from body tissue in which it is implanted or on which it is worn comprises a ceramic housing, preferably of alumina, having a housing sidewall extending to an annular rim surrounding a housing opening leading into the housing. A housing ferrule sealed to the annular rim of the ceramic housing has a ferrule opening.

A printed circuit board (PCB) assembly contained inside the ceramic housing comprises a printed circuit board supporting at least one electronic component that is configured to control functions of the active medical device. At least one electrical termination of the printed circuit board is electrically connected to the electronic component. Preferably, the printed circuit board has a plurality of electrical terminations and the insulator of the GTMS supports a plurality of terminal pins. One of the plurality of terminal pins is connected to one of the plurality of electrical terminations on the insulative substrate.

Further, a rechargeable electrical power source supported on the printed circuit board or positioned adjacent to the PCB is electrically connected to the at least electronic component. The electrical power source is selected from a capacitor, an alkaline cell, a primary lithium cell, a rechargeable lithium-ion cell, a Ni/cadmium cell, a Ni/metal hydride cell, a supercapacitor, and a thin film solid-state cell with a rechargeable chemistry being preferred.

The PCB assembly also has a charging coil connected to a charging circuit. The charging circuit is configured to convert RF or inductive energy received from the charging coil into a direct current voltage to charge the rechargeable electrical power source to power the at least one electronic component of the PCB assembly.

The active medical device of the present invention also has a glass-to-metal seal (GTMS). The GTMS has a GTMS ferrule with a ferrule opening. An insulator comprising an insulator sidewall hermetically sealed to the GTMS ferrule in the ferrule opening extends to an insulator device side spaced from an insulator body fluid side. At least one via hole extends through the insulator to the device and body fluid sides, and at least one terminal pin is hermetically sealed to the insulator in the via hole. The terminal pin has a proximal or device side portion that extends outwardly beyond the insulator device side and a distal or body fluid side portion that extends outwardly beyond the insulator body fluid side. Then, with the terminal pin device side portion of the GTMS being electrically connected to the at least one electrical termination of the printed circuit board of the PCB assembly, the GTMS ferrule is sealed to the housing ferrule so that the GTMS closes the housing opening of the ceramic housing.

These and other aspects of the present invention will become increasingly more apparent to those skilled in the art by reference to the following detailed description and to the appended drawings.

### BRIEF DESCRIPTION OF THE DRAWINGS

FIG. 1 is a wire formed diagram of a generic human body showing a number of medical devices 100A to 100L according to the present invention that can either be implanted in a patient's body tissue or attached externally to the body.

FIG. 2 is a simplified block diagram of an exemplary medical system 10 according to various embodiments of the present invention.

FIG. 3 is an exploded view of an active medical device 12 according to the present invention including a PCB assembly 40 housed inside a ceramic housing 22 and connected to a glass-to-metal seal 50 closing the open end of the housing.

FIG. 4 is a side elevational view of the PCB assembly 40 connected to the GTMS 50 shown in FIG. 3.

FIG. 5 is a perspective view showing the PCB assembly 40 connected to the GTMS 50 in FIG. 4 being moved into the ceramic housing 22 shown in FIG. 3.

FIG. 6 is a side elevational view, partly in cross-section, shown the ferrule 52 comprising the GTMS 50 shown in FIGS. 4 and 5 after having been welded to the device ferrule 28 connected to the ceramic housing 22 housing the PCB assembly 40.

### DETAILED DESCRIPTION OF THE PREFERRED EMBODIMENTS

As described in the present specification, the term “active medical device” refers to a medical device that is powered by an electrical power source and that can be implanted in a patient's body tissue or worn externally on the body. The active medical device is configured to either provide electrical stimulation to body tissue in which it is implanted or to body tissue on which it is externally worn, or to sense biological signals from adjacent body tissue in which it is implanted or from body tissue on which it is externally worn. The active medical device can also be configured to both provide electrical stimulation and sense biological signals to and from body tissue in which it is implanted or on which it is worn.

Further, as described in the present specification, a printed circuit board (PCB) assembly is comprised of a PCB as a flexible and insulative substrate supporting at least one electronic component connected to at least two electrically-conductive and spaced-apart traces. The PCB can be bent and twisted into unique shapes with the substrate maintaining the at least two conductive traces electrically isolated from each other. The opposed ends of the conductive traces are connected to bond pads or electrical terminations supported on the insulative substrate or the traces are connected to conductive eyelets in the substrate. The bond pads or conductive eyelets are configured to electrically connect to terminal pins extending from a glass-to-metal seal (GTMS) of the medical device and to the casing for the electrical power source. With the exception of the connection bond pads or the conductive eyelets, the PCB is insulated so that the electrical traces cannot short to the electrical power source and to the medical device housing.

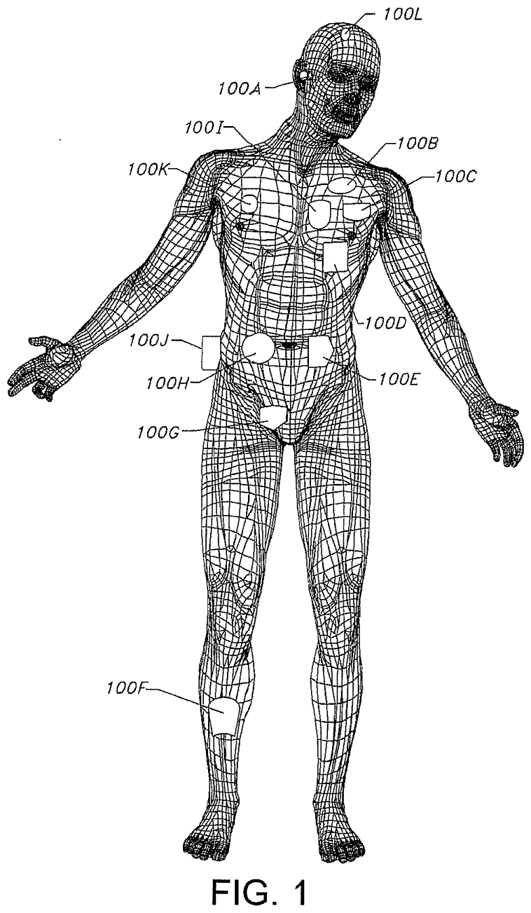

Turning now to the drawings, FIG. 1 is a wire form diagram of a generic human body illustrating various types of active implantable and external medical devices according to the present invention that can either be implanted in a patient's body or attached externally to the body.

Numerical designation 100A represents a family of hearing devices which can include the group of cochlear implants, piezoelectric sound bridge transducers, and the like.

Numerical designation 100B represents a variety of neurostimulators, brain stimulators, and brain sensors. Neurostimulators are used to stimulate the Vagus nerve, for example, to treat epilepsy, obesity, and depression. Brain stimulators are pacemaker-like devices and include electrodes implanted deep into the brain for sensing the onset of a seizure and also for providing electrical stimulation to brain tissue to prevent a seizure from actually occurring. If present, lead wires associated with a deep brain stimulator are often placed using real time MRI imaging. Sensors include optical sensors, motion sensors, acoustic sensors, pressure sensors, analyte sensors, and electromagnetic sensors, among others.

Numerical designation 100C shows a cardiac pacemaker which is well-known in the art.

Numerical designation 100D includes the family of left ventricular assist devices (LVADs) and artificial heart devices.

Numerical designation 100E includes a family of drug pumps which can be used for dispensing insulin, chemotherapy drugs, pain medications, and the like.

Numerical designation 100F includes a variety of bone growth stimulators for rapid healing of fractures.

Numerical designation 100G includes urinary incontinence devices.

Numerical designation 100H includes the family of pain relief spinal cord stimulators and anti-tremor stimulators. Numerical designation 100H also includes an entire family of other types of neurostimulators used to block pain.

Numerical designation 100I includes both implantable cardioverter defibrillator (ICD) devices and congestive heart failure devices (CHF). These are known in the art as cardio resynchronization therapy devices, otherwise known as CRT devices.

Numerical designation 100J illustrates an externally worn pack. The pack could be an external insulin pump, an external drug pump, an external neurostimulator or even a ventricular assist device.

Numerical designation 100K illustrates one of various types of EKG/ECG external skin electrodes which can be placed at various external locations on the body.

Numerical designation 100L represents external EEG electrodes that are placed on the head.

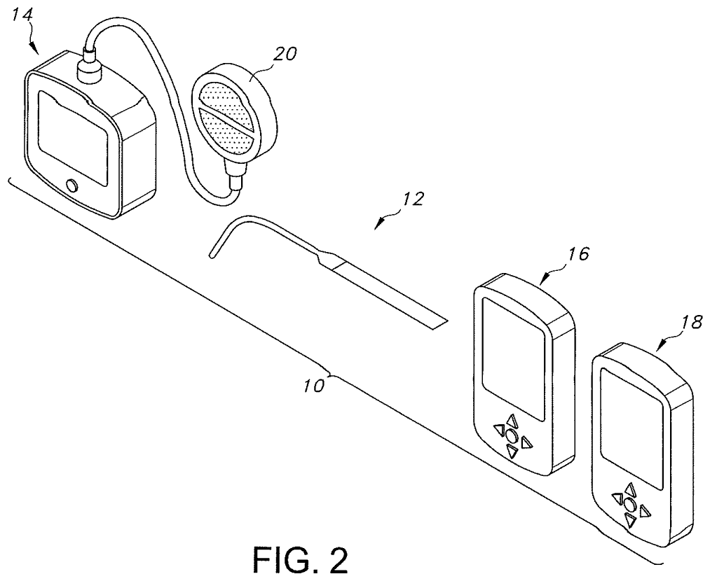

To provide context to the various active medical devices 100A to 100L illustrated in FIG. 1, FIG. 2 illustrates a simplified block diagram of an exemplary medical system 10 according to the present invention. The medical system 10 includes an active medical device 12, which is any one of the various medical devices 100A to 100L illustrated in FIG. 1 and which can be implanted in a patient's body tissue or worn externally on the body. In that respect, while the active medical device 12 is shown as an elongate device, the shape of the medical device 12 is not limited to the elongate shape that is shown. For example, the active medical device 12 can have a cylindrical shape or a shape that is not elongated.

The medical system 10 also has an external charger 14, a patient programmer 16, and a clinician programmer 18. The patient programmer 16 and the clinician programmer 18 may be portable handheld devices, such as a smartphone or other custom device, that are used to configure the active medical device 12 so that the medical device can operate in a desired manner. The patient programmer 16 is used by the patient in whom the active medical device 12 is implanted or on whom the active medical device is externally worn. The patient may adjust the parameters of electrical stimulation delivered by the active medical device 12, such as by selecting a stimulation program, changing the amplitude and frequency of the electrical stimulation, among other parameters, and by turning stimulation on and off. Additionally, the patient programmer 16 may collect and or display data being collected by the active medical device 12 and alert the patient to potential health risks.

The clinician programmer 18 is used by medical personnel to configure the other system components and to adjust stimulation parameters that the patient is not permitted to control. These include setting up stimulation programs among which the patient may choose and setting upper and lower limits for the patient's adjustments of amplitude, frequency, and other parameters. Although FIG. 2 illustrates the patient programmer 16 and the clinician programmer 18 as two separate devices, they may be integrated into a single programmer in some embodiments.

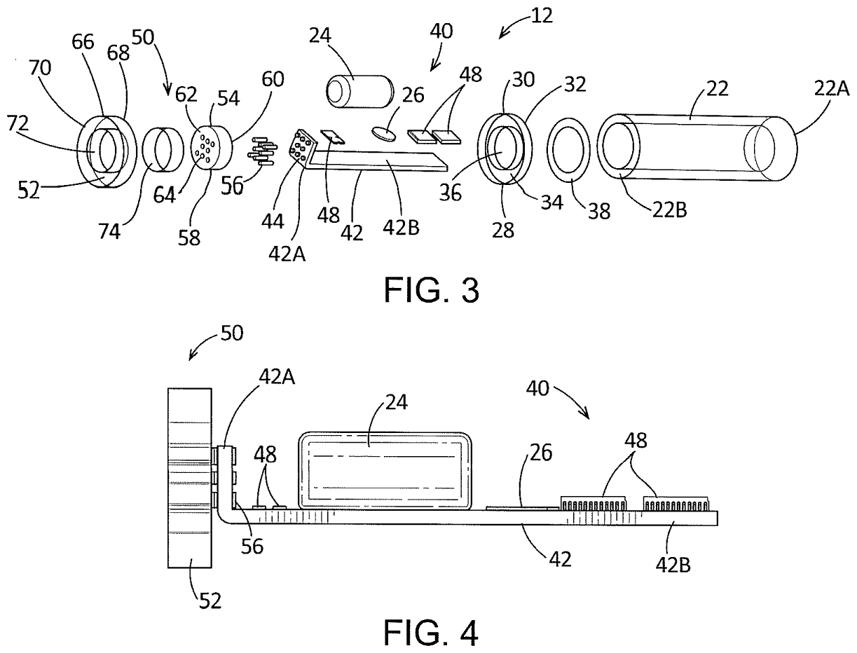

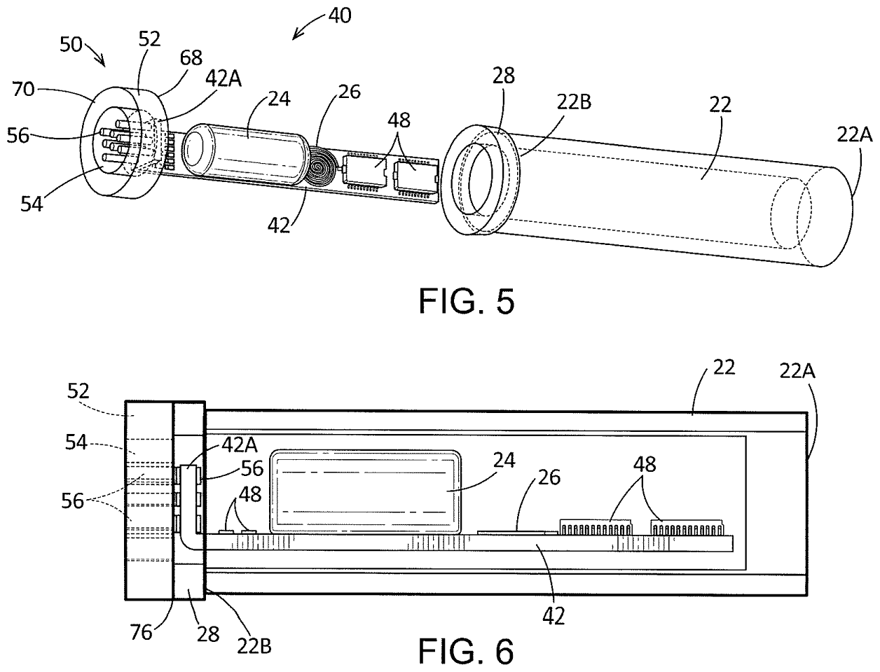

Electrical power may be delivered to the active medical device 12 through an external charging pad 20 that is connected to the external charger 14. The external charging pad 20 is configured to charge a rechargeable electrical power source 24 (FIGS. 3 to 6) through a charging coil 26 connected to a charging circuit (not shown) of the active medical device 12.

The external charging pad 20 can be a hand-held device that is connected to the external charger 14, or it can be an internal component of the external charger. The external charger 14 and the charging pad 20 can also be integrated into a single device that is strapped on or attached to the patient with adhesive.

Referring now to FIGS. 3 to 6, these drawings illustrate an exemplary embodiment of the active medical device 12 shown in the exemplary medical system 10 (FIG. 2) according to the present invention that can be either implanted in a patient's body tissue or worn externally on the body. The active medical device 12 is comprised of a ceramic housing 22. An essentially pure alumina is a preferred ceramic material. The term “essentially pure alumina” refers to an alumina ceramic having the chemical formula Al₂O₃. “Essentially pure” means that the post-sintered alumina is at least 96% alumina. In a preferred embodiment, the post-sintered alumina is at least 99% high purity alumina.

The ceramic housing 22 has a surrounding sidewall extending from a closed bottom 22A to a proximal annular rim 22B. The proximal rim 22B surrounds and defines an opening leading into the housing 22. An essentially high purity alumina is preferred for the housing 22 including its rim 22B.

A housing ferrule 28, preferably made from titanium, is hermetically sealed to the housing rim 22B. The housing ferrule 28 is an annular ring-shaped member comprising an outer cylindrically-shaped sidewall 30 extending to a proximal ring-shaped surface 32 spaced from a distal ring-shaped surface 34. The proximal and distal ring-shaped surfaces 32, 34 surround a cylindrically-shaped opening 36.

The housing ferrule 28 is different than a feedthrough ferrule, which will be discussed in detail hereinafter.

The housing ferrule 28 is hermetically secured to the proximal annular rim 22B of the ceramic housing 22 by a brazing process in which the rim 22B is provided with a metallization (not shown). A suitable metallization comprises two metallization layers, a first adhesion layer that is directly applied to the outwardly facing edge of the rim 22B, and a second, wetting layer, which is applied on top of the adhesion layer. In a preferred embodiment, the adhesion layer is titanium, and the wetting layer is either molybdenum or niobium. However, for the sake of simplicity, the adhesion and wetting layers are intentionally not shown.

A gold ring-shaped preform 38 is seated on the metallized proximal rim 22B. Then, the proximal ring-shaped surface 32 of the housing ferrule 28 is contacted to the gold preform 38. The housing ferrule 28 contacting the gold pre-form 38 contacting the metallized rim 22B of the ceramic housing 22 as an assembly is then subjected to a brazing process including a heating protocol which is sufficient to melt the gold preform 38, as is well known by those skilled in the art related to brazing a ceramic material to a metallic ferrule. The molten gold then wets to the metallization contacting the proximal rim 22B of the ceramic housing 22 and to the housing ferrule 28. The heating portion of the brazing process is then discontinued, and the gold is allowed to cool and solidify to thereby form a hermetic seal joining the housing ferrule 28 to the ceramic housing 22 at the proximal annular rim 22B.

A printed circuit board (PCB) assembly 40 resides inside the ceramic housing 22. The PCB assembly 40 is comprised of a printed circuit board or substrate 42 that extends from a proximal end 42A to a distal portion 42B. The proximal end 42A of the printed circuit board 42 supports at least two, and preferably, a plurality of proximal electrical terminations 44. The printed circuit board 42 also supports the electrical power source 24 and at least one, and preferably a plurality of electronic components 48 that are connected to the proximal electrical terminations 44. The electronic components 48 are powered by the electrical power source 24 and electrically connected to the electrical terminations 44 by circuit traces (not shown). Alternately, the electrical power source 24 is not supported on the PCB 42 but is positioned adjacent to the PCB to power the electronic components 48.

The PCB assembly 40 controls the various functions performed by the active medical device 12. These include, but are not limited to, providing electrical stimulation to body tissue in which the active medical device 12 is implanted or to body tissue on which it is externally worn, and receiving sensed biological signals pertaining to functions of the body tissue in which the active medical device 12 is implanted or receiving sensed biological signals from body tissue on which it is externally worn. The active medical device 12 can also be configured to both provide electrical stimulation and sense biological signals to and from body tissue in which it is implanted or on which it is worn.

Preferably, the PCB assembly 40 also supports the previously described charging coil 26 connected to a charging circuit (not shown). The charging circuit is configured to convert RF or inductive energy signals received by the inductive charging coil 26 from the external charging pad 20 connected to the external charger 14 (FIG. 2) into a direct current voltage to charge the electrical power source 24 to power the electronic components 48 of the PCB assembly 40. It is the power source 24 electrically connected to the electronic components 48 which makes the medical device an active medical device 12.

The electrical power source 24 can be a capacitor or a rechargeable electrochemical cell, for example a hermetically sealed rechargeable Li-ion electrochemical cell. However, the electrical power source 24 is not limited to any one chemistry or even a rechargeable chemistry and can be of an alkaline cell, a primary lithium cell, a rechargeable lithium-ion cell, a Ni/cadmium cell, a Ni/metal hydride cell, a supercapacitor, a thin film solid-state cell, and the like. Preferably, the electrical power source 24 is a lithium-ion electrochemical cell comprising a carbon-based or Li₄Ti₅O₁₂-based anode and a lithium metal oxide-based cathode, such as of LiCoO₂ or lithium nickel manganese cobalt oxide (LiNiₐMn_{b}Co_{1-a-b}O₂). The electrical power source 24 can also be a solid-state thin film electrochemical cell having a lithium anode, a metal-oxide based cathode and a solid electrolyte, such as an electrolyte of LiPON (LiₓPO_{y}N_{z}).

As shown in FIGS. 3 to 6, a glass-to-metal seal (GTMS) 50 is hermetically secured to the housing ferrule 28 to close the proximal open end of the ceramic housing 22. The GTMS 50 is comprised of the previously discussed feedthrough ferrule 52 which supports an insulator 54 that seals between the ferrule 52 and at least one, and preferably a plurality of terminal pins 56. The insulator 54 is preferably of a ceramic material such as of alumina and comprises an outer sidewall 58 that extends to a device side 60 spaced from a body fluid side 62. The height of the outer sidewall 58 between the device and body fluid sides 60, 62 defines the thickness of the insulator 54.

The insulator 54 is further provided with at least one and preferably a plurality of via holes 64, and the at least one and preferably the plurality of terminal pins 56 are received in the via holes 64 in the insulator 54. Preferably, there are as many terminal pins 56 as there are via holes 64. The terminal pins 56 are made of molybdenum, aluminum, nickel alloy, or stainless steel, the former being preferred.

In a similar manner as the previously described housing ferrule 28, the ferrule 52 for the GTMS 50 is preferably made from titanium and is an annular ring-shaped member comprising an outer cylindrically-shaped sidewall 66 extending to a proximal ring-shaped surface 68 spaced from a distal ring-shaped surface 70. The proximal and distal ring-shaped surfaces 68, 70 surround a cylindrically-shaped opening 72.

The insulator 54 is hermetically sealed to the GTMS ferrule 52 in its cylindrically-shaped opening 72 and to the at least one, and preferably, the plurality of terminal pins 56. In that respect, the via holes 64 and the outer sidewall 58 of the insulator 54 are provides with a metallization in a similar manner as previously described for hermetically sealing the housing ferrule 28 to the rim 22B of the ceramic housing 22. A gold braze 74 hermetically seals the insulator 54 to the GTMS ferrule 52 and a ring-shaped gold preform (not shown) hermetically seal each of the terminal pins 56 to the insulator 54 in the via holes 64. The hermetically sealed terminal pins have a sufficient length so that their device side portion extends outwardly beyond the device side 60 of the insulator 54 and their body fluid side portion extends outwardly beyond the body fluid side 62 of the insulator 54.

The active medical device 12 shown in the medical system 10 illustrated in FIG. 2 and which is representative of the various active medical device 100A to 100L depicted in FIG. 1 is hermetically sealed by moving the PCB assembly 40 through the open housing ferrule 28 sealed to the rim 22B of the ceramic housing 22. With the PCB assembly 40 housed inside the ceramic housing 22, the device side 68 of the ferrule 52 for the GTMS 50 is seated against the body fluid side 34 of the housing ferrule 28.

The electrical terminations 44 at the proximal end of the printed circuit board 42 of the PCB assembly 40 can be a conductive eyelet or a bond pad that is connected to a respective one of the terminal pins 56 of the GTMS 50. In particular, the proximal or device side portion of the terminal pin 56 is either received in the conductive eyelet or connected to the bond pad. The electronic components 48 powered by the electrical power source 24 and electrically connected to the electrical terminations 44 are now electrically connected to the terminal pins 56. The ferrules 28 and 52 are then hermetically secured to each other by a weld 76 (FIG. 6), preferably a laser weld. In that manner, the GTMS 50 hermetically closes the open proximal end of the ceramic housing 22 connected to the housing ferrule 28 and prevents body fluids, and the like, from contacting the PCB assembly 40 including the charging coil 26 connected to the charging circuit, power source 26 and electronic components 48.

The hermetically sealed active medical device 12 of the present invention is connectable to a conventional molded header (not shown) supporting a plurality of terminal blocks. In that configuration, the distal or body fluid side portions of the terminal pins 56 of the GTMS 50 are connected to the terminal blocks in the header. The terminal blocks are detachably connectable to a lead to provide both stimulation and sensing capability to body tissue in which electrodes of the lead are implanted. The present active medical device 12 is also configured to connect to the exemplary connectors operably attached to stimulation elements, as described in U.S. Pat. No. 11,097,096 to Linden et al., which is assigned to Nalu Medical, Inc., Carlsbad, CA.

Thus, the present invention describes an active medical device that can be implantable or worn externally and that is detachably connectable to a lead to provide both stimulation and sensing capability. The active medical device has a housing that is made from a ceramic material, preferably alumina, and is preferably powered by a rechargeable electrical power source that is connected to a charging circuit connected to an inductive charging coil contained inside the ceramic housing. The charging circuit is configured to convert RF or inductive energy signals received by the inductive charging coil from an external charger into a direct current voltage to charge the electrical power source to power the electronic components of a PCB assembly contained inside the device housing. Moving the charging coil out of the header as is common in conventional active medical devices and inside the ceramic housing saves a substantial amount of space in the molded header. A smaller medical device is easier to implant in a patient and would be expected to cause less trauma to the patient. A smaller medical device is also expected to be less bothersome to a patient.

It is appreciated that various modifications to the inventive concepts described herein may be apparent to those skilled in the art without departing from the spirit and scope of the present invention as defined by the hereinafter appended claims.

1. An active medical device, comprising:
a) a ceramic housing comprising a housing sidewall extending to an annular rim surrounding a housing opening leading into the housing;
b) a housing ferrule having a ferrule opening, wherein the housing ferrule is sealed to the annular rim of the ceramic housing;
c) a printed circuit board (PCB) assembly contained inside the ceramic housing and comprising a printed circuit board supporting at least one electronic component that is configured to control functions of the active medical device, wherein the printed circuit board is provided with at least one electrical termination connected to the at least one electronic component;
d) an electrical power source contained inside the ceramic housing and being electrically connected to the at least one electronic component of the PCB assembly;
e) a ceramic-to-metal seal, comprising:
i) a feedthrough ferrule having a feedthrough ferrule opening;
ii) an insulator comprising an insulator sidewall extending to an insulator device side spaced from an insulator body fluid side, wherein the insulator sidewall is hermetically sealed to the feedthrough ferrule in the feedthrough ferrule opening; and
iii) at least one terminal pin hermetically sealed to the insulator, the terminal pin comprising a terminal pin device side portion that extends outwardly beyond the insulator device side and a terminal pin body side fluid portion that extends outwardly beyond the insulator body fluid side,
f) wherein the feedthrough ferrule is sealed to the housing ferrule so that the ceramic-to-metal seal closes the housing opening of the ceramic housing with the terminal pin device side portion of the ceramic-to-metal seal being electrically connected to the at least one electrical termination of the printed circuit board of the PCB assembly.

2. (canceled)

3. The active medical device of claim 1, further comprising a charging coil connected to a charging circuit contained inside the ceramic housing, wherein the electrical power source is rechargeable, and wherein the charging circuit is configured to convert RF or inductive energy received from the charging coil into a direct current voltage to charge the rechargeable electrical power source to power the at least one electronic component of the PCB assembly.

4. The active medical device of claim 1, wherein the feedthrough ferrule and the housing ferrule are made of titanium, and wherein the feedthrough ferrule is welded to the housing ferrule.

5. The active medical device of claim 1, wherein the printed circuit board is provided with a plurality of electrical terminations and the insulator of the ceramic-to-metal seal supports a plurality of terminal pins, and wherein one of the plurality of terminal pins is connected to one of the plurality of electrical terminations on the printed circuit board.

6. The active medical device of claim 5, wherein the plurality of electrical terminations are selected from a conductive eyelet and a bond pad, and the device side portion of the plurality of terminal pins is either received in the conductive eyelet or connected to the bond pad.

7. The active medical device of claim 1, wherein the ceramic housing is comprised of alumina.

8. The active medical device of claim 1, wherein the electrical power source is selected from a capacitor, an alkaline cell, a primary lithium cell, a rechargeable lithium-ion cell, a Ni/cadmium cell, a Ni/metal hydride cell, a supercapacitor, and a thin film solid-state cell.

9. The active medical device of claim 1, configured to both provide electrical stimulation and sense biological signals to and from body tissue in which it is implanted or on which it is worn.

10. The active medical device of claim 1, wherein the medical device is selected from a cochlear implant, a piezoelectric sound bridge transducer, a neurostimulator, a brain stimulator, a brain sensor, a neurostimulator configured to stimulate the Vagus nerve, an optical sensor, a motion sensor, an acoustic sensor, a pressure sensor, an analyte sensor, an electromagnetic sensor, a cardiac pacemaker, a left ventricular assist device, an artificial heart device, an insulin drug pump, a chemotherapy drug pump, a pain medication pump, a bone growth stimulator, a urinary incontinence device, a spinal cord stimulator, an anti-tremor stimulator, an implantable cardioverter defibrillator, a congestive heart failure device, an external insulin pump, an external drug pump, and an external neurostimulator, an external ventricular assist device.

11. An active medical device, comprising:
a) an alumina housing comprising a housing sidewall extending to an annular rim surrounding a housing opening leading into the housing;
b) a housing ferrule having a ferrule opening, wherein the housing ferrule is sealed to the annular rim of the ceramic housing;
c) a printed circuit board (PCB) assembly contained inside the ceramic housing and comprising:
i) a printed circuit board supporting at least one electronic component that is configured to control functions of the active medical device;
ii) at least one electrical termination of the printed circuit board connected to the at least one electronic component;
iii) a rechargeable electrical power source electrically connected to the at least one electronic components and
iv) a charging coil connected to a charging circuit, wherein the charging circuit is configured to convert RF or inductive energy received from the charging coil into a direct current voltage to charge the rechargeable electrical power source to power the at least one electronic component of the PCB assembly; and
d) a ceramic-to-metal seal, comprising:
i) a feedthrough ferrule having a feedthrough ferrule opening;
ii) an insulator comprising an insulator sidewall extending to an insulator device side spaced from an insulator body fluid side, wherein the insulator sidewall is hermetically sealed to the feedthrough ferrule in the feedthrough ferrule opening; and
at least one terminal pin hermetically sealed to the insulator, the terminal pin comprising a terminal pin device side portion that extends outwardly beyond the insulator device side and a terminal pin body fluid side portion that extends outwardly beyond the insulator body fluid side,
e) wherein the feedthrough ferrule is sealed to the housing ferrule so that the ceramic-to-metal seal closes the housing opening of the ceramic housing with the terminal pin device side portion of the ceramic-to-metal seal being electrically connected to the at least one electrical termination of the printed circuit board of the PCB assembly.

12. The active medical device of claim 11, wherein the printed circuit board is provided with a plurality of electrical terminations and the insulator of the ceramic-to-metal seal supports a plurality of terminal pins, and wherein one of the plurality of terminal pins is connected to one of the plurality of electrical terminations on the printed circuit board.

13. The active medical device of claim 11, wherein the plurality of electrical terminations are selected from a conductive eyelet and a bond pad, and the device side portion of the plurality of terminal pins is either received in the conductive eyelet or connected to the bond pad.

14-16. (canceled)

17. The active medical device of claim 11, wherein the PCB assembly is configured to communicate with an external device to at least one of adjust electrical stimulation delivered to body tissue or sense biological signals from body tissue in which the active medical device is implanted or on which it is worn.

18. An active medical device, comprising:
a) a ceramic housing comprising a housing sidewall extending to an annular rim surrounding a housing opening leading into the housing;
b) a housing ferrule having a ferrule opening, wherein the housing ferrule is sealed to the annular rim of the ceramic housing;
c) a printed circuit board (PCB) assembly contained inside the ceramic housing and comprising a printed circuit board supporting at least one electronic component that is configured to control functions of the active medical device, wherein the printed circuit board is provided with at least one electrical termination connected to the at least one electronic component;
d) a capacitor contained inside the ceramic housing and being electrically connected to the at least one electrical termination connected to the at least one electronic component of the PCB assembly;
a ceramic-to-metal seal, comprising:
i) a feedthrough ferrule having a feedthrough ferrule opening;
ii) an insulator comprising an insulator sidewall extending to an insulator device side spaced from an insulator body fluid side, wherein the insulator sidewall is hermetically sealed to the feedthrough ferrule in the feedthrough ferrule opening; and
iii) at least one terminal pin hermetically sealed to the insulator, the terminal pin comprising a terminal pin device side portion that extends outwardly beyond the insulator device side and a terminal pin body side fluid portion that extends outwardly beyond the insulator body fluid side,
f) wherein the feedthrough ferrule is sealed to the housing ferrule so that the ceramic-to-metal seal closes the housing opening of the ceramic housing with the terminal pin device side portion of the ceramic-to-metal seal being electrically connected to the at least one electrical termination of the printed circuit board of the PCB assembly.

19. The active medical device of claim 18, further comprising a charging coil connected to a charging circuit contained inside the ceramic housing, wherein the charging circuit is configured to convert RF or inductive energy received from the charging coil into a direct current voltage to charge the capacitor to power the at least one electronic component of the PCB assembly.

20. The active medical device of claim 18, wherein the feedthrough ferrule and the housing ferrule are made of titanium, and wherein the feedthrough ferrule is welded to the housing ferrule.

21. The active medical device of claim 18, wherein the printed circuit board is provided with a plurality of electrical terminations and the insulator of the ceramic-to-metal seal supports a plurality of terminal pins, and wherein one of the plurality of terminal pins is connected to one of the plurality of electrical terminations on the printed circuit board.

22. The active medical device of claim 21, wherein the plurality of electrical terminations are selected from a conductive eyelet and a bond pad, and the device side portion of the plurality of terminal pins is either received in the conductive eyelet or connected to the bond pad.

23. The active medical device of claim 18, wherein the ceramic housing is comprised of alumina,

24. The active medical device of claim 18, wherein the medical device is selected from a cochlear implant, a piezoelectric sound bridge transducer, a neurostimulator, a brain stimulator, a brain sensor, a neurostimulator configured to stimulate the Vagus nerve, an optical sensor, a motion sensor, an acoustic sensor, a pressure sensor, an analyte sensor, an electromagnetic sensor, a cardiac pacemaker, a left ventricular assist device, an artificial heart device, an insulin drug pump, a chemotherapy drug pump, a pain medication pump, a bone growth stimulator, a urinary incontinence device, a spinal cord stimulator, an anti-tremor stimulator, an implantable cardioverter defibrillator, a congestive heart failure device, an external insulin pump, an external drug pump, and an external neurostimulator, an external ventricular assist device.
